(19) United States
(12) Patent Application Publication
(10) Pub. No.: US 2014/0066945 A1
(43) Pub. Date: Mar. 6, 2014

(54) SURGICAL IMPLANT SYSTEM AND METHOD

(71) Applicants: Kevin R. Humphreys, Oxford, MS (US); C. Renee Diaz, Olive Branch, MS (US)

(72) Inventors: Kevin R. Humphreys, Oxford, MS (US); C. Renee Diaz, Olive Branch, MS (US)

(73) Assignee: Warsaw Orthopedic, Inc., Warsaw, IN (US)

(21) Appl. No.: 13/600,643

(22) Filed: Aug. 31, 2012

(51) Int. Cl. A61B 17/88

(52) U.S. Cl. 606/104

(57) ABSTRACT

A surgical implant driver includes a member defining a longitudinal axis and including an engagement portion. The engagement portion includes a first surface and a second surface disposed in a serial configuration along the longitudinal axis. The first surface includes a drive interface engageable with a first surface of an implant fastener and the second surface includes at least a retention interface engageable with a second surface of the implant fastener. Systems and methods are disclosed.

### TECHNICAL FIELD

The present disclosure generally relates to medical devices for the treatment of musculoskeletal disorders, and more particularly to a surgical system for delivering and/or fastening implants with a surgical site and a method for treating a spine.

### BACKGROUND

Spinal pathologies and disorders such as scoliosis and other curvature abnormalities, kyphosis, degenerative disc disease, disc herniation, osteoporosis, spondylolisthesis, stenosis, tumor, and fracture may result from factors including trauma, disease and degenerative conditions caused by injury and aging. Spinal disorders typically result in symptoms including deformity, pain, nerve damage, and partial or complete loss of mobility.

Non-surgical treatments, such as medication, rehabilitation and exercise can be effective, however, may fail to relieve the symptoms associated with these disorders. Surgical treatment of these spinal disorders includes correction, fusion, fixation, discectomy, laminectomy and implantable prosthetics. As part of these surgical treatments, implants such as bone fasteners, connectors, plates and vertebral rods are often used to provide stability to a treated region. These implants can redirect stresses away from a damaged or defective region while healing takes place to restore proper alignment and generally support the vertebral members. For example, rods and plates may be attached via the fasteners to the exterior of two or more vertebral members. This disclosure describes an improvement over these prior art technologies.

### SUMMARY

In one embodiment, in accordance with the principles of the present disclosure, a surgical implant driver is provided. The surgical implant driver includes a member defining a longitudinal axis and including an engagement portion. The engagement portion includes a first surface and a second surface disposed in a serial configuration along the longitudinal axis. The first surface includes a drive interface engageable with a first surface of an implant fastener and the second surface includes at least a retention interface engageable with a second surface of the implant fastener.

### BRIEF DESCRIPTION OF THE DRAWINGS

The present disclosure will become more readily apparent from the specific description accompanied by the following drawings, in which:

FIG. 1 is a perspective view of one particular embodiment of components of a surgical implant system in accordance with the principles of the present disclosure;

FIG. 2 is a break away perspective view of a component of the surgical implant system shown in FIG. 1;

FIG. 3 is an end view of the component shown in FIG. 2;

FIG. 4 is a break away perspective view of a component of the surgical implant system shown in FIG. 1;

FIG. 5 is a cutaway perspective view of the component shown in FIG. 4;

FIG. 6 is a break away perspective view of the components of the surgical implant system shown in FIG. 1;

FIG. 7 is a cross section view of the components shown in FIG. 6;

FIG. 8 is a break away perspective view of one embodiment of a component of the surgical implant system shown in FIG. 1;

FIG. 9 is an end view of the component shown in FIG. 8;

FIG. 10 is a break away perspective view of one embodiment of the spinal implant system including the component shown in FIG. 8; and

FIG. 11 is a cross section view of the components shown in FIG. 10.

Like reference numerals indicate similar parts throughout the figures.

### DETAILED DESCRIPTION

The exemplary embodiments of a surgical implant system are discussed in terms of medical devices for the treatment of musculoskeletal disorders and more particularly, in terms of a surgical implant system for delivering and/or fastening implants with a surgical site and a method for treating a spine. In one embodiment, the system includes a surgical implant driver and a bone screw. In one embodiment, the surgical implant driver has a step design defining a dual drive feature configured to prevent stripping of the bone screw by distributing torque load across at least two features. It is envisioned that the dual drive feature can reduce and/or eliminate the likelihood of stripping the bone screw during insertion or extraction.

In one embodiment, the system includes an instrument driver and/or bone fastener having a step design that prevents stripping of the bone fastener by distributing the torque load across a drive interface and a retention interface. In one embodiment, the driver includes a smaller thickness portion that drives the bone fastener while a larger thickness portion drives and retains the bone fastener.

In one embodiment, the system includes a driver and/or fastener having a smaller hexalobe that provides driving torque and a larger hexalobe that provides driving torque and self retention of the fastener. In one embodiment, the retention feature includes a twisted configuration. In one embodiment, the retention feature includes a tapered configuration. The system provides a dual drive configuration, which can reduce and/or eliminate stripping a fastener head during insertion or extraction of implants.

In one embodiment, the system includes a T10-T8 step hexalobe configuration such that the bone fastener and/or driver has a T10 driving/retention surface and a T8 driving surface. In one embodiment, the T10 driving/retention surface includes a twisted surface that provides retention at each contact surface. In one embodiment, the T10 driving/retention surface includes a tapered surface that provides retention at a transition line.

It is envisioned that the surgical implant system of the present disclosure may be employed to treat spinal disorders such as, for example, degenerative disc disease, disc herniation, osteoporosis, spondylolisthesis, stenosis, scoliosis and other curvature abnormalities, kyphosis, tumor and fractures. It is contemplated that the surgical implant system of the present disclosure may be employed with other osteal and bone related applications, including those associated with diagnostics and therapeutics. It is further contemplated that the disclosed surgical implant system may be alternatively employed in a surgical treatment with a patient in a prone or supine position, and/or employ various surgical approaches to the spine, including anterior, posterior, posterior mid-line, lateral, postero-lateral, and/or antero-lateral approaches, and in other body regions. The surgical implant system of the present disclosure may also be alternatively employed with procedures for treating the lumbar, cervical, thoracic, sacral and pelvic regions of a spinal column. The surgical implant system of the present disclosure may also be used on animals, bone models and other non-living substrates, such as, for example, in training, testing and demonstration.

The surgical implant system of the present disclosure may be understood more readily by reference to the following detailed description of the embodiments taken in connection with the accompanying drawing figures, which form a part of this disclosure. It is to be understood that this application is not limited to the specific devices, methods, conditions or parameters described and/or shown herein, and that the terminology used herein is for the purpose of describing particular embodiments by way of example only and is not intended to be limiting. Also, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure. For example, the references “upper” and “lower” are relative and used only in the context to the other, and are not necessarily “superior” and “inferior”.

Further, as used in the specification and including the appended claims, “treating” or “treatment” of a disease or condition refers to performing a procedure that may include administering one or more drugs to a patient (human, normal or otherwise or other mammal), employing implantable devices, and/or employing instruments that treat the disease, such as, for example, microdiscectomy instruments used to remove portions bulging or herniated discs and/or bone spurs, in an effort to alleviate signs or symptoms of the disease or condition. Alleviation can occur prior to signs or symptoms of the disease or condition appearing, as well as after their appearance. Thus, treating or treatment includes preventing or prevention of disease or undesirable condition (e.g., preventing the disease from occurring in a patient, who may be predisposed to the disease but has not yet been diagnosed as having it). In addition, treating or treatment does not require complete alleviation of signs or symptoms, does not require a cure, and specifically includes procedures that have only a marginal effect on the patient. Treatment can include inhibiting the disease, e.g., arresting its development, or relieving the disease, e.g., causing regression of the disease. For example, treatment can include reducing acute or chronic inflammation; alleviating pain and mitigating and inducing re-growth of new ligament, bone and other tissues; as an adjunct in surgery; and/or any repair procedure. Also, as used in the specification and including the appended claims, the term “tissue” includes soft tissue, ligaments, tendons, cartilage and/or bone unless specifically referred to otherwise.

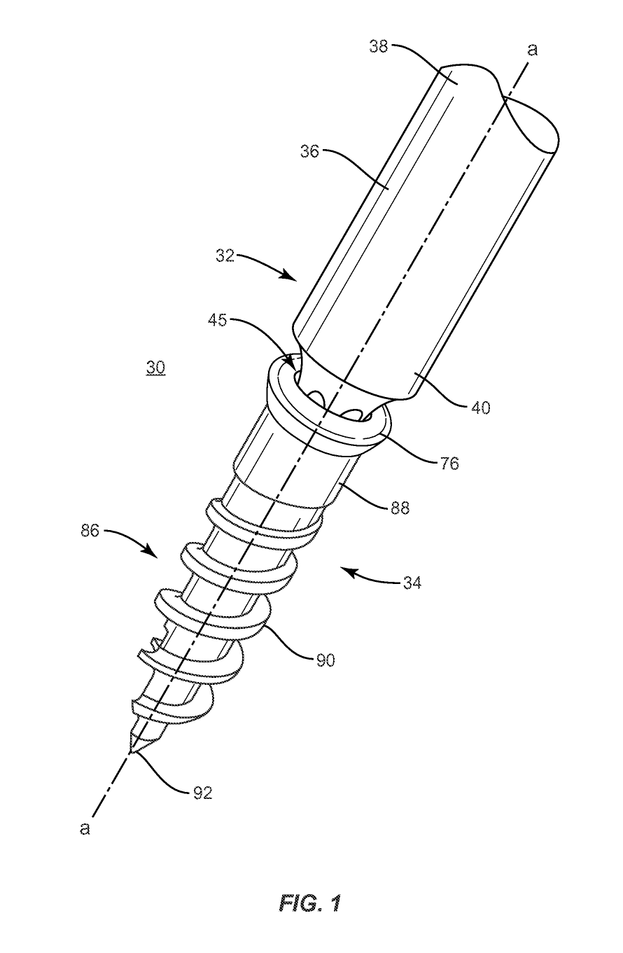

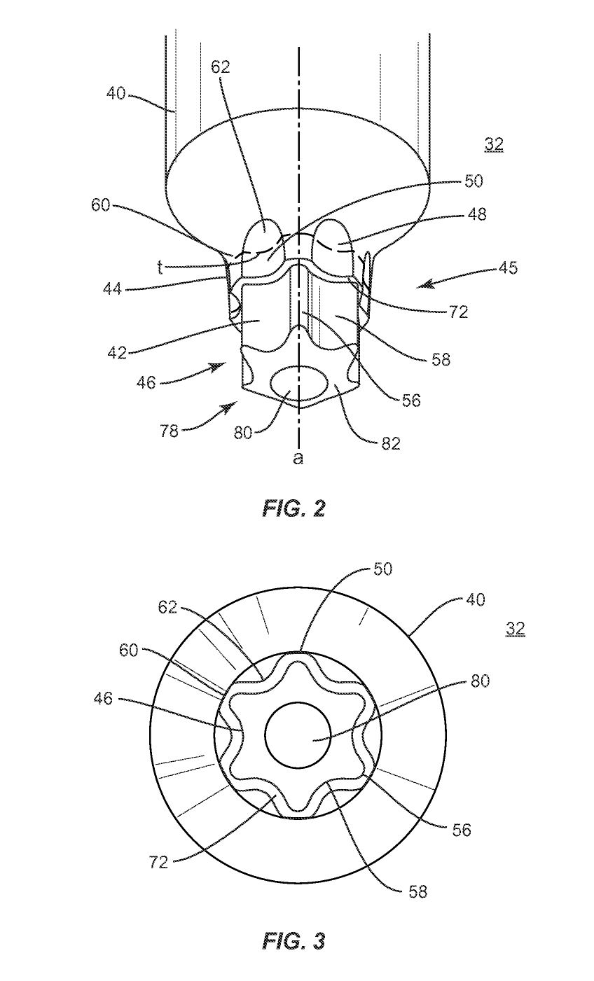

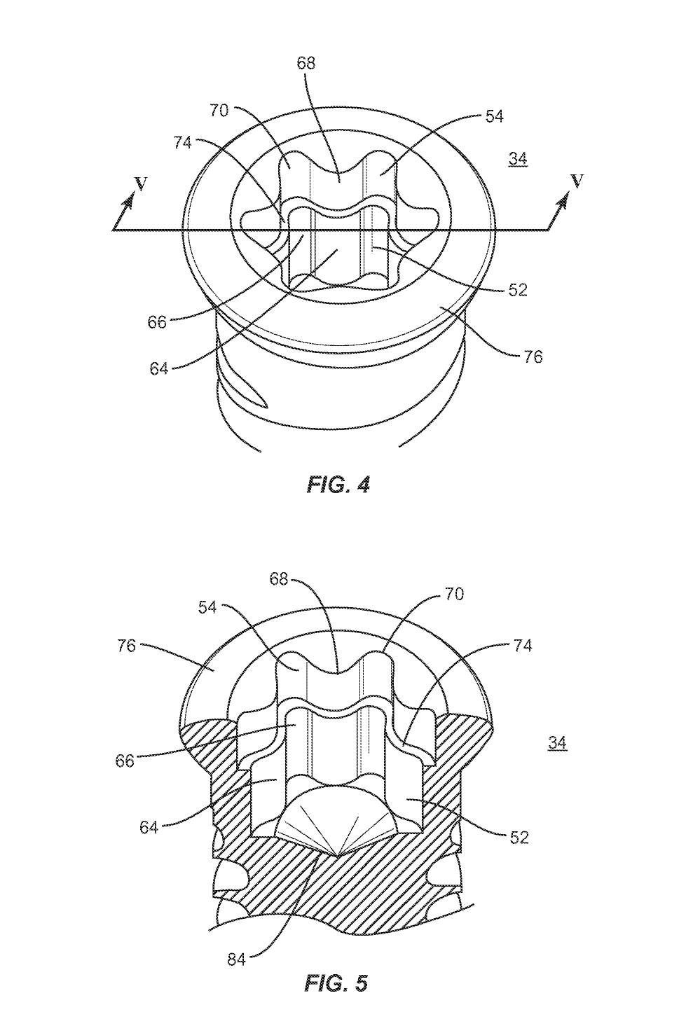

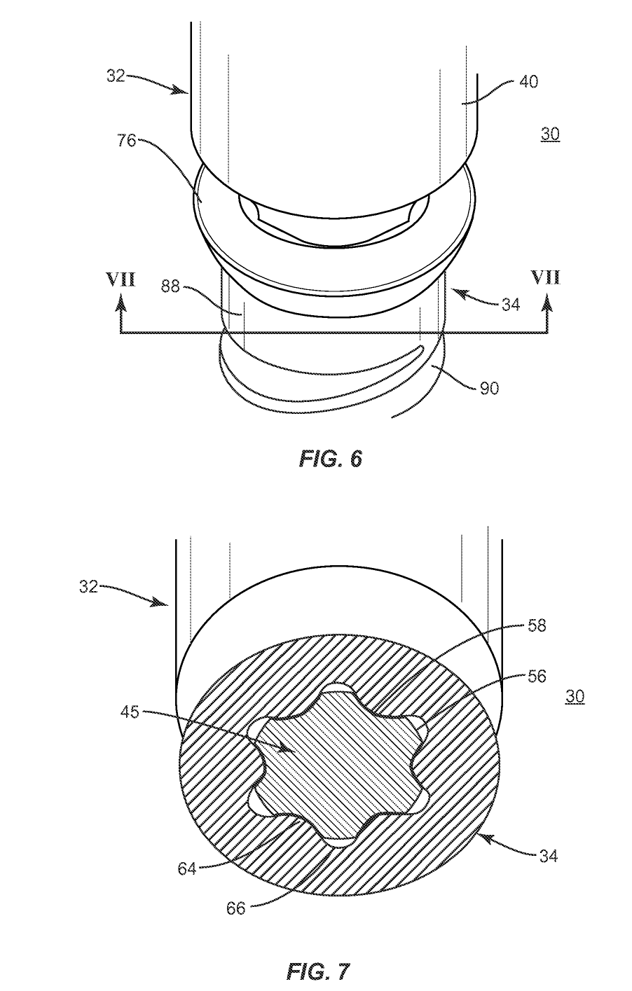

The following discussion includes a description of a surgical implant system including a driver and a bone fastener, related components and methods of employing the surgical implant system in accordance with the principles of the present disclosure. Alternate embodiments are also disclosed. Reference will now be made in detail to the exemplary embodiments of the present disclosure, which are illustrated in the accompanying figures. Turning now to FIGS. 1-7, there is illustrated components of a surgical implant system 30 including a screw driver, such as, for example, a surgical implant driver 32 and an implant fastener, such as, for example, a bone screw 34, in accordance with the principles of the present disclosure.

The components of surgical implant system 30 can be fabricated from biologically acceptable materials suitable for medical applications, including metals, synthetic polymers, ceramics and bone material and/or their composites, depending on the particular application and/or preference of a medical practitioner. For example, the components of surgical implant system 30, individually or collectively, can be fabricated from materials such as stainless steel alloys, commercially pure titanium, titanium alloys, Grade 5 titanium, super-elastic titanium alloys, cobalt-chrome alloys, stainless steel alloys, superelastic metallic alloys (e.g., Nitinol, super elasto-plastic metals, such as GUM METAL® manufactured by Toyota Material Incorporated of Japan), ceramics and composites thereof such as calcium phosphate (e.g., SKELITE™ manufactured by Biologix Inc.), thermoplastics such as polyaryletherketone (PAEK) including polyetheretherketone (PEEK), polyetherketoneketone (PEKK) and polyetherketone (PEK), carbon-PEEK composites, PEEK-BaSO₄ polymeric rubbers, polyethylene terephthalate (PET), fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyamide, polyimide, polyetherimide, polyethylene, epoxy, bone material including autograft, allograft, xenograft or transgenic cortical and/or corticocancellous bone, and tissue growth or differentiation factors, partially resorbable materials, such as, for example, composites of metals and calcium-based ceramics, composites of PEEK and calcium based ceramics, composites of PEEK with resorbable polymers, totally resorbable materials, such as, for example, calcium based ceramics such as calcium phosphate, tri-calcium phosphate (TCP), hydroxyapatite (HA)-TCP, calcium sulfate, or other resorbable polymers such as polyaetide, polyglycolide, polytyrosine carbonate, polycaroplaetohe and their combinations. Various components of surgical implant system 30 may have material composites, including the above materials, to achieve various desired characteristics such as strength, rigidity, elasticity, compliance, biomechanical performance, durability and radiolucency or imaging preference. The components of surgical implant system 30, individually or collectively, may also be fabricated from a heterogeneous material such as a combination of two or more of the above-described materials. The components of surgical implant system 30 may be monolithically formed, integrally connected or include fastening elements and/or instruments, as described herein.

Spinal implant system 30 is employed, for example, with an open or mini-open, minimal access and/or minimally invasive including percutaneous surgical technique to deliver and fasten an implant, such as, for example, a bone fastener at a surgical site within a body of a patient, for example, a section of a spine. In one embodiment, the components of spinal implant system 30 are configured to fix a spinal rod, connector and/or plate to a spine via a bone fastener for a surgical treatment to treat various spine pathologies, such as those described herein.

Driver 32 includes a member, such as, for example, an elongated shaft 36 and extends between a first end, such as, for example, a proximal end 38 and a second end, such as, for example, a distal end 40 including an engagement portion 45. Shaft 36 has a cylindrical cross sectional configuration between ends 38, 40. It is envisioned that all or only a portion of shaft 36 may have alternate cross section configurations, such as, for example, oval, oblong, triangular, square, polygonal, irregular, uniform, non-uniform, offset, staggered, and/or tapered. Driver 32 defines a longitudinal axis a. Driver 32 is tapered between end 40 and portion 45 such that portion 45 has a width that is less than end 40.

Portion 45 includes an outer surface defining a first surface 42 and a second surface 44 disposed in a serial configuration along axis a, with surface 42 being distal to surface 44. Surface 42 defines a first drive interface 46 and surface 44 includes a proximal portion that defines a retention interface 48 and a distal portion that defines a second drive interface 50. Interfaces 48, 50 are separated by a transition line t extending transverse to axis a. Interface 48 has a first diameter and interface 50 has a second diameter that is less than the first diameter.

Surface 42 includes a plurality of spaced apart axial teeth, such as, for example, a plurality of spaced apart lobes 56 and a plurality of recesses 58 between lobes 56 that define a first hexalobe configuration. It is contemplated that surface 42 may include one or a plurality of spaced apart axial teeth.

Surface 44 includes a plurality of spaced apart axial teeth, such as, for example, a plurality of spaced apart lobes 60 and a plurality of recesses 62 between lobes 60 that define a second hexalobe configuration. It is contemplated that surface 44 may include one or a plurality of spaced apart axial teeth. It is envisioned that surfaces 42, 44 may include any number of lobes 56, 60 and/or recesses 58, 62. Lobes 56 are aligned with lobes 60 and recesses 58 are aligned with recesses 62. It is contemplated that surfaces 42, 44 may have alternate configurations, such as, for example, thread forms, triangular, square, polygonal, star, torx, irregular, uniform, non-uniform, offset, staggered, and/or tapered.

Each lobe 60 has a width defined by the distance between recesses 62 that tapers between a proximal end of surface 44 and a distal end of surface 44 such that the width of each lobe 60 is greater at the proximal end of surface 44 than at the distal end of surface 44. Each recess 62 has a width between lobes 60 that tapers between the proximal end of surface 44 and the distal end of surface 44 such that the width of each recess 62 is greater at the distal end of surface 44 than at the proximal end of surface. Each recess 62 is concavely curved at the proximal end of each recess.

The distance between opposite lobes 56 is less than the distance between opposite lobes 60 and the distance between opposite recesses 58 is less than the distance between opposite recesses 62 such that the serial configuration of surfaces 42, 44 includes a stepped orientation. In one embodiment, surface 44 has a width between opposite recesses 62 that is between about 5% and about 25% greater than a width of surface 42 between opposite recesses 58 and a width between opposite lobes 60 that is between about 5% and about 25% greater than a width of surface 42 between opposite lobes 56. In one embodiment, surface 44 has a width between opposite recesses 62 that is between about 10% and about 20% greater than a width of surface 42 between opposite recesses 58 and a width between opposite lobes 60 that is between about 10% and about 20% greater than a width of surface 42 between opposite lobes 56. In one embodiment, surface 44 has a width between opposite recesses 62 that is about 15% greater than a width of surface 42 between opposite recesses 58 and a width between opposite lobes 60 that is about 15% greater than a width of surface 42 between opposite lobes 56.

Surface 44 is tapered between the proximal end of surface 44 and the distal end of surface 44 such that the width of surface 44 between opposite lobes 60 at the proximal end of surface 44 is greater than the width of surface 44 between opposite lobes 60 at the distal end of surface 44 to facilitate engagement of driver 32 with screw 34. Each recess 62 is tapered between line t and the proximal end of each recess 62 such that each recess 62 has a depth that is greater at line t than at the proximal end of each recess 62. The tapered portions of surface 44 and recesses 62 define interface 48 and facilitate engagement of driver 32 with screw 34 and to retain driver 32 with screw 34 once engaged with one another.

In one embodiment, a height of interface 46 is defined by a height of recesses 58 extending along surface 42, and/or a combined height of interfaces 48, 50 is defined by a height of recesses 62 extending along surface 44. It is contemplated that the height of interface 46 may be greater than, less than or substantially equal to the combined height of interfaces 48, 50.

Interface 46 is engageable with a first surface 52 of screw 34 and interfaces 48, 50 are engageable with a second surface 68, 54 of screw 34. Surfaces 52, 54 define an inner surface of a head 76 of screw 34. Surfaces 52, 54 are disposed in a serial configuration along axis a, with surface 54 being distal to surface 52. Surface 52 has a height that is substantially equivalent to the combined height of interfaces 48, 50 and surface 54 has a height that is substantially equivalent to the height of interface 46.

Surface 52 has a hexalobe socket configuration and defines a plurality of lobes 64 and a plurality of recesses 66 between lobes 64. Surface 54 has a hexalobe socket configuration and defines a plurality of lobes 68 and a plurality of recesses 70 between lobes 68. Lobes 64 and recesses 66 define a first hexalobe socket engageable with interface 46 and lobes 68 and recesses 70 define a second hexalobe socket engageable with interfaces 48, 50. Lobes 64 are aligned with lobes 68 and recesses 66 are aligned with recesses 70. It is envisioned that surfaces 52, 54 may include any number of lobes 64, 68 and/or recesses 66, 70. It is further envisioned that surfaces 52, 54 may have alternate configurations, such as, for example, thread form, triangular, square, polygonal, star, torx, irregular, uniform, non-uniform, offset, staggered, and/or tapered.

The distance between opposite lobes 64 is less than the distance between opposite lobes 68 and the distance between opposite recesses 66 is likewise less than the distance between opposite recesses 70 such that the serial configuration of surfaces 52, 54 includes a stepped orientation. In one embodiment, surface 54 has a width between opposite recesses 70 that is between about 5% and about 25% greater than a width of surface 52 between opposite recesses 66 and a width between opposite lobes 68 that is between about 5% and about 25% greater than a width of surface 52 between opposite lobes 64. In one embodiment, surface 54 has a width between opposite recesses 70 that is between about 10% and about 20% greater than a width of surface 52 between opposite recesses 66 and a width between opposite lobes 68 that is between about 10% and about 20% greater than a width of surface 52 between opposite lobes 64. In one embodiment, surface 54 has a width between opposite recesses 70 that is about 15% greater than a width of surface 52 between opposite recesses 66 and a width between opposite lobes 68 that is about 15% greater than a width of surface 52 between opposite lobes 64.

When driver 32 is matingly engaged with screw 34, interface 46 engages surface 52 such that lobes 56 engage recesses 66 and lobes 64 engage recesses 58 and interfaces 48, 58 engage surface 54 such that lobes 60 engage recesses 70 and lobes 68 engage recesses 62. The number of lobes 56 is equal to the number of recesses 66, the number of lobes 64 is equal to the number of recesses 58, the number of lobes 60 is equal to the number of recesses 70 and the number of lobes 68 is equal to the number of recesses 62.

An interface between surfaces 42, 44 define a lip 72 configured to engage a ledge 74 of screw 34 defined by an interface between surfaces 52, 54. Lip 72 and ledge 74 each extend transverse to axis a and each have a planar configuration. Lip 72 defines a step between surfaces 42, 44 and ledge 74 defines a step between surfaces 52, 54 such that lip 72 and ledge 74 define the stepped orientations of surfaces 42, 44 and surfaces 52, 54. It is envisioned that lip 72 and ledge 74 may be disposed through angular ranges in various orientations relative to axis a, such as, for example, transverse, perpendicular and/or other angular orientations such as acute or obtuse. It is further envisioned that lip 72 and/or ledge 74 may have alternate surface configurations to enhance engagement with one another such as, for example, rough, arcuate, undulating, dimpled and/or textured, according to the requirements of a particular application.

Interface 46 includes a tapered portion 78 between distal ends of lobes 56 and recesses 58 and a distal face 80 of interface 46 defined by a beveled edge 82. The inner surface of screw 34 includes a chamfered bottom surface 84 configured to engage portion 78 when driver 32 is engaged with screw 34. It is envisioned that surface 84 may include a beveled edge having a configuration similar to that of edge 82 and a distal face similar to that of face 80 such that the beveled edge of surface 84 engages edge 82 and the distal face of surface 84 engages face 80 when driver 32 is engaged with screw 34.

Screw 34 includes a shaft 86 configured to penetrate tissue, such as, for example, bone. Shaft 86 includes an outer surface having a smooth first portion 88 adjacent head 76 and a second portion 90 distal to portion 88 having an external thread form. Shaft 86 is tapered between head 76 and a distal tip 92 of portion 90. Head 76 has a maximum width that is greater than a maximum width of any portion of shaft 86. The maximum width of head 76 is substantially equivalent to a maximum width of shaft 36. The thread form on the outer surface of portion 90 is configured to penetrate tissue, such as, for example, bone, to fix screw 34 in such tissue. It is contemplated that the thread form on the outer surface of portion 44 may extend through portion 88 such that shaft 86 is threaded along the entire length thereof. It is further contemplated that all or only a portion of portion 90 may have various surface configurations, such as, for example, rough, threaded, arcuate, undulating, porous, semi-porous, dimpled, polished and/or textured according to the requirements of a particular application.

In assembly, operation and use, an implant system, similar to system 30 described herein, is employed with a surgical procedure for treatment of a spinal disorder affecting a section of a spine of a patient, as discussed herein. For example, the spinal implant system can be used with a surgical procedure for treatment of a condition or injury of an affected section of the spine including vertebrae (not shown). It is contemplated that one or all of the components of system 30 can be delivered or implanted as a pre-assembled device or can be assembled in situ. System 30 may be completely or partially revised, removed or replaced.

For example, spinal implant system 30 can be employed with a surgical treatment of an applicable condition or injury of an affected section of a spinal column and adjacent areas within a body, such as, for example, vertebrae. It is envisioned that spinal implant system 30 may be employed with one or a plurality of vertebra. To treat a selected section of the vertebrae, a medical practitioner obtains access to a surgical site including the vertebrae in any appropriate manner, such as through incision and retraction of tissues. It is envisioned that spinal implant system 30 can be used in any existing surgical method or technique including open surgery, mini-open surgery, minimally invasive surgery and percutaneous surgical implantation, whereby the vertebrae are accessed through a mini-incision, or sleeve that provides a protected passageway to the area. Once access to the surgical site is obtained, the particular surgical procedure can be performed for treating the spine disorder.

An incision is made in the body of a patient and a cutting instrument (not shown) creates a surgical pathway for implantation of components of spinal implant system 30. A preparation instrument (not shown) can be employed to prepare tissue surfaces of the vertebrae, as well as for aspiration and irrigation of a surgical region according to the requirements of a particular surgical application.

Pilot holes or the like are made in selected vertebra of the vertebrae for receiving bone screws 34. System 30 is disposed adjacent the vertebrae at a surgical site and the components of system 30 including driver 32, are manipulable to drive, torque, insert or otherwise connect bone screws 34 to the vertebrae, according to the particular requirements of the surgical treatment. For example, portion 45 is inserted into an opening defined by the inner surface of head 76 such that lobes 56 are disposed along recesses 70 and lobes 68 are disposed along recesses 58. Driver 32 is translated axially relative to screw 34 along axis a in a first direction such that lobes 56 are positioned within recesses 66 such that lobes 64 engage recesses 58. As lobes 56 are positioned within recesses 66, lobes 68 enter recesses 62 at distal ends of recesses 62 and lobes 60 are positioned within recesses 70. Driver 32 is further translated axially relative to screw 34 along axis a in the first direction such that lobes 68 cross line t and engage the tapered portions of recesses 62. As lobes 68 engage the tapered portions of recesses 62, lip 72 engages ledge 74 and edge 82 engages surface 84 to retain driver 32 with screw 34.

Driver 32 is matingly and releasably fixed with screw 34. Interface 46 engages surface 52 in a driving interface configuration to provide a driving torque to fasten screw 34 with the vertebrae. Interface 50 engages surface 54 in a driving interface configuration to provide a driving torque to fasten screw 34 with the vertebrae. Interface 48 engages surface 54 in a retention interface configuration to releasably retain screw 34 with driver 32.

Once access to the surgical site is obtained, the particular surgical procedure is performed. The components of system 30, including screw 34 are employed to augment the surgical treatment. For example, screw 34 may be inserted into bone or other tissue with driver 32, for example via clockwise or counterclockwise rotation. Screw 34 may be delivered, introduced, inserted and/or removed from the bone or other tissue with driver 32. Upon completion of a surgical procedure, driver 32 may be disengaged from screw 34, and the non-implanted components, including driver 32 may be removed from the surgical site and the incision closed.

It is contemplated one or a plurality of bone fasteners may be employed with a single vertebral level. It is further contemplated that the bone fasteners may be engaged with vertebrae in various orientations, such as, for example, series, parallel, offset, staggered and/or alternate vertebral levels. Spinal implant system 30 can be used with various bone fasteners, mono-axial screws, pedicle screws or multi-axial screws used in spinal surgery.

In one embodiment, spinal implant system 30 includes an agent, which may be disposed, packed or layered within, on or about the components and/or surfaces of spinal implant system 30. It is envisioned that the agent may include bone growth promoting material, such as, for example, bone graft to enhance fixation of the bone fasteners with the vertebrae. It is contemplated that the agent may include one or a plurality of therapeutic agents and/or pharmacological agents for release, including sustained release, to treat, for example, pain, inflammation and degeneration. The components of spinal implant system 30 can be made of radiolucent materials such as polymers. Radiomarkers may be included for identification under x-ray, fluoroscopy, CT or other imaging techniques. It is envisioned that the use of microsurgical and image guided technologies may be employed to access, view and repair spinal deterioration or damage, with the aid of spinal implant system 30.

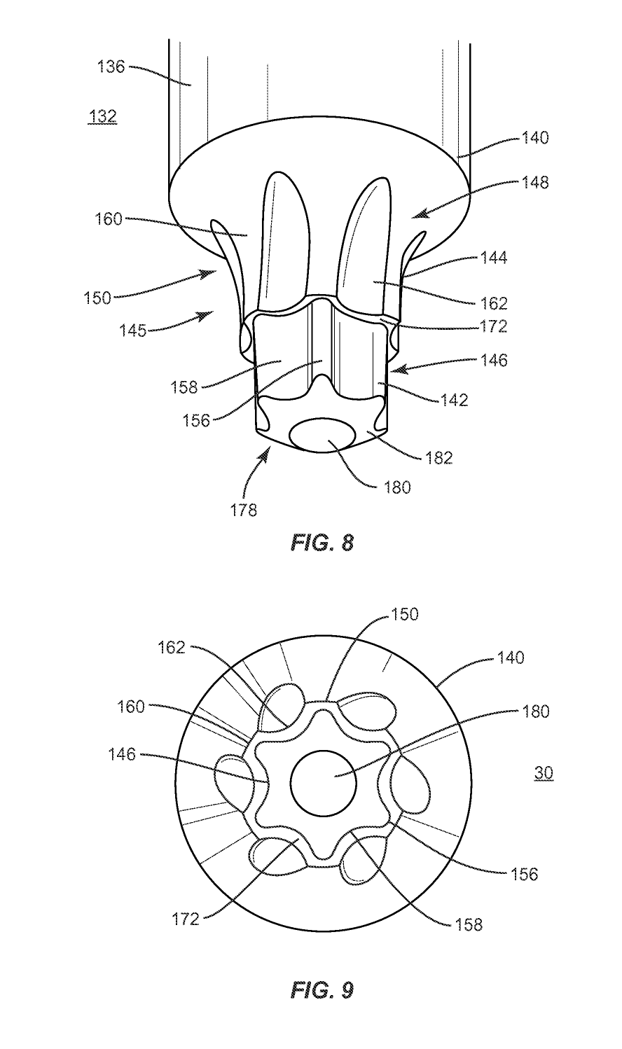

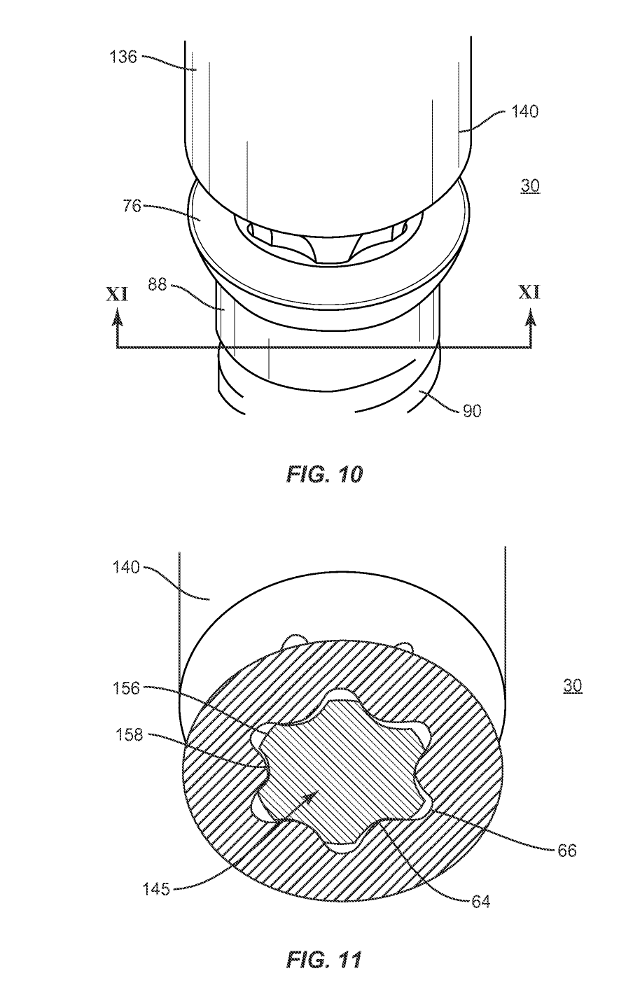

In one embodiment, as shown in FIGS. 8-11, system 30, similar to the systems and methods described above with regard to FIGS. 1-7, includes screw 34 and a driver 132, similar to driver 32. Driver 132 extends along a longitudinal axis a1 and includes an elongated shaft 136 having a distal end 140 including an engagement portion 145. Portion 145 includes an outer surface defining a first surface 142 and a second surface 144 disposed in a serial configuration along axis a1, with surface 142 being distal to surface 144. Surface 142 defines a first drive interface 146 and surface 144 includes a proximal portion that defines a retention interface 148 and a distal portion that defines a second drive interface 150. Interface 148 has a first diameter and interface 150 has a second diameter that is less than the first diameter. Interface 146 is engageable with surface 52 and interfaces 148, 150 are engageable with surface 54.

Surface 142 includes a plurality of spaced apart linear lobes 156 and a plurality of recesses 158 between lobes 156 that define a first hexalobe configuration. Surface 144 includes a plurality of helical teeth, such as, for example, helical lobes 160 and a plurality of helical recesses 162 between lobes 160 that define a second hexalobe configuration. Lobes 156 are aligned with lobes 160 and recesses 158 are aligned with recesses 162. When driver 132 is engaged with screw 34, interface 146 engages surface 52 such that lobes 156 engage recesses 66 and lobes 64 engage recesses 158. Interfaces 148, 150 engage surface 54 such that lobes 160 engage recesses 70 and lobes 68 engage recesses 162.

Surface 144 is tapered between a proximal end of surface 144 and a distal end of surface 144 such that the width of surface 144 between opposite lobes 160 at the proximal end of surface 144 is greater than the width of surface 144 between opposite lobes 160 at the distal end of surface 144. Each recess 162 is tapered between a distal end and a proximal end of each recess 162 such that each recess 162 has a depth that is greater at the distal end than at the proximal end of each recess 162. The helical portions of lobes 160 and recesses 162 define interface 148 and facilitate engagement of driver 132 with screw 34 and to retain driver 132 with screw 34 once engaged with one another. The helical orientation of lobes 160 provide a twisted configuration of interface 148 such that a retaining engagement with surface 54 is defined axially along each lobe 160.

An interface between surfaces 142, 144 defines a lip 172 configured to engage ledge 74. Lip 172 extends transverse to axis a1 and has a planar configuration. Lip 172 defines a step between surfaces 142, 144 such that lip 172 contributes to the stepped orientation of surfaces 142, 144.

Interface 146 includes a tapered portion 178 between distal ends of lobes 156 and recesses 158 and a distal face 180 of interface 146 defined by a beveled edge 182. Surface 84 engages edge 182 when driver 132 is engaged with screw 34.

In use, portion 145 is inserted into the opening in head 76 such that lobes 156 are disposed along recesses 70 and lobes 68 are disposed along recesses 158. Driver 132 is translated axially relative to screw 34 along axis a1 in a first direction such that lobes 156 are positioned within recesses 66 and lobes 64 engage recesses 158. As lobes 156 are positioned within recesses 66, lobes 68 enter recesses 162 at distal ends of recesses 162 and lobes 160 are positioned within recesses 70. The helical configuration of lobes 160 engage the wall surface of lobes 68 in a retaining engagement with surface 54, which is defined axially along each lobe 160. This configuration provides a retaining interface between driver 132 and screw 34. Driver 132 is further translated axially relative to screw 34 along axis a1 in the first direction such that lobes 68 engage the tapered portions of recesses 162. As lobes 68 engage the tapered portions of recesses 162, lip 172 engages ledge 74 and edge 182 engages surface 84 to retain driver 132 with screw 34. Driver 132 is retained and releasably engaged with screw 34 for fastening with tissue, as described above. Driver 132 may be disengaged from screw 34.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. A surgical implant driver comprising:
a member defining a longitudinal axis and including an engagement portion;
the engagement portion including a first surface and a second surface disposed in a serial configuration along the longitudinal axis,
the first surface including a drive interface engageable with a first surface of an implant fastener and the second surface including at least a retention interface engageable with a second surface of the implant fastener.

2. A surgical implant driver as recited in claim 1, wherein the member including an elongated shaft extending between a first end and a second end, the second end including the engagement portion.

3. A surgical implant driver as recited in claim 1, wherein the serial configuration includes a stepped orientation of the first and second surfaces of the engagement portion.

4. A surgical implant driver as recited in claim 1, wherein the first surface of the engagement portion includes a first diameter and the second surface of the engagement portion includes a distal portion having a second diameter greater than the first diameter and a proximal portion having a third diameter that is greater than the second diameter.

5. A surgical implant driver as recited in claim 1, wherein the drive interface includes at least one axial tooth.

6. A surgical implant driver as recited in claim 1, wherein the retention surface includes at least one axial tooth.

7. A surgical implant driver as recited in claim 1, wherein the retention surface includes a plurality of spaced apart axial teeth.

8. A surgical implant driver as recited in claim 1, wherein the retention interface includes a plurality of helical teeth.

9. A surgical implant driver as recited in claim 1, wherein the retention interface includes a plurality of axial teeth disposed in an axially tapered configuration.

10. A surgical implant driver as recited in claim 1, wherein the retention interface includes a plurality of axial teeth, the teeth including a distal portion having a first diameter and a proximal portion having a second diameter that is greater than the first diameter.

11. A surgical implant driver as recited in claim 1, wherein at least one of the drive interface and the retention interface includes a hexalobe configuration.

12. A surgical implant driver as recited in claim 1, wherein the second surface includes a plurality of helical teeth such that the retention interface is axially oriented along the helical teeth.

13. A surgical implant driver as recited in claim 1, wherein the second surface includes a proximal portion having a first diameter and a distal portion having a second diameter that is less than the first diameter, the proximal portion including the retention interface and the distal portion including a drive interface.

14. A surgical implant driver as recited in claim 1, wherein the first and second surfaces of the implant fastener are disposed in a serial configuration including a stepped orientation.

15. A surgical implant system comprising:
a driver defining a longitudinal axis and a first drive interface and a retention interface disposed in a serial configuration along the longitudinal axis; and
an implant fastener including a first drive interface and a retention interface disposed in a serial configuration,
wherein the retention interfaces are engageable to retain the implant fastener with the driver and the first drive interfaces are engageable to drive the implant fastener into tissue.

16. A surgical implant system as recited in claim 15, wherein the retention interface includes a plurality of axial teeth, the teeth including a distal portion having a first diameter and a proximal portion having a second diameter that is greater than the first diameter.

17. A surgical implant system as recited in claim 15, wherein the second surface includes a plurality of helical teeth such that the retention interface is axially oriented along the helical teeth.

18. A surgical implant system as recited in claim 15, wherein the second surface includes a proximal portion having a first diameter and a distal portion having a second diameter that is less than the first diameter, the proximal portion including the retention interface and the distal portion including a drive interface.

19. A surgical implant system as recited in claim 15, wherein the first drive interface has a first hexalobe configuration and the retention interface has a second hexalobe configuration.

20. A surgical implant system comprising:
a screw driver including an elongated shaft and extending between a proximal end and a distal end, the screw driver defining a longitudinal axis,
the distal end including a first surface having a first hexalobe configuration and a second surface having a second hexalobe configuration disposed in a serial configuration along the longitudinal axis,
the first surface including a first drive interface and the second surface including a retention interface and a second drive interface; and
a bone screw including a head and a shaft extending therefrom configured for penetrating tissue, the head defining a first hexalobe socket being engageable with the first drive interface and a second hexalobe socket being engageable with the second drive interface and the retention interface.
